(19) United States
(12) Patent Application Publication
(10) Pub. No.: US 2022/0395353 A1
(43) Pub. Date: Dec. 15, 2022

(54) DEVICE FOR OROFACIAL PHOTOGRAPHY

(71) Applicant: David GALLER, Morrisville, NC (US)

(72) Inventor: David GALLER, Morrisville, NC (US)

(21) Appl. No.: 17/742,869

(22) Filed: May 12, 2022

Related U.S. Application Data
(60) Provisional application No. 63/210,110, filed on Jun. 14, 2021.

(51) Int. Cl. A61C 5/90; A61B 1/24; A61B 5/00

(52) U.S. Cl. CPC A61C 5/90; A61B 1/24; A61B 5/0088

(57) ABSTRACT

The instant disclosure describes a novel dental appliance and methods of use thereof for taking orofacial photos.

### RELATED APPLICATIONS

This application claims priority from U.S. Provisional Application No. 63/210,110 filed on Jun. 14, 2021, which is herein incorporated by reference in its entirety.

### FIELD OF THE INVENTION

The present disclosure relates to the field of orthodontics, and more particularly to the design of a dental appliance for facilitating orofacial photography of the teeth.

### BACKGROUND OF THE INVENTION

Traditional dental and orthodontic treatment involves one or a series of patient visits to the dentist for treatment, or to monitor and adjust long-term treatment during a given treatment period. Such visits include an examination of the patient's teeth to determine what, if any, progress was made since the last visit and to determine how treatment is tailored to the patient. Examination of the patient's teeth may include not only a physical exam, but also photography of the patient's teeth to track progress.

An advancement in the alternatives to traditional braces such as preformed appliances and aligners for positioning teeth makes use of modern day computer technology. Customized treatment plans may be designed using computer modeling and software to understand and monitor the three-dimensional configuration of a patient's teeth both before and during the treatment process. Rather than fixed appliances such as braces, the technology allows for a series of custom designed, successive aligners for the patient to wear over the teeth to exert the required force to facilitate tooth movement as the dentist sees fit. The aligners are to be worn for an extended period during the day, but are also removable to facilitate better oral hygiene as compared to traditional braces.

A further advantage of modern day technology is the ability to reduce office visits and create virtual treatment plans to observe and monitor the patients teeth due to the widespread use and availability of both photographic and video technology. With the average cell phone camera, a patient may obtain the necessary photos of their own teeth to send to theft provider, who may then use the images to measure and design the next set of aligners in the patient's treatment plan and simply mail them directly to the patient. Such a model is not only convenient and efficient, but provides the required safety and social-distancing that is of concern during the current pandemic, while also allowing patients to successfully continue their orthodontic treatment. However, this “self-photography” requires the patient to use one hand to ensure lips are fully retracted so full tooth alignment is visible while also holding the cell phone camera in the other hand to obtain images. An appliance that retracts the lips to provide an unimpeded view of the teeth without patient support would allow the patient to use both hands to ensure full and complete images of the teeth are obtained.

Thus, there is a need for dental appliances that are easy for patients to use and that may assist them in self-imaging their teeth during long-term dental treatment.

### SUMMARY OF THE INVENTION

One embodiment described here is an appliance comprising: an upper lip retractor; a lower lip retractor; a hinge spring; wherein the hinge spring exerts an upward force on the upper lip retractor and a downward force on the lower lip retractor.

In one aspect, the upper lip retractor is in the shape of an upper dental arch. In another aspect, the upper dental arch has a width of about 40 mm to about 75 mm. In another aspect, the upper dental arch has a radius of about 20 mm to about 35 mm. In another aspect, the upper lip retractor comprises a well for receiving the upper lip. In another aspect, the upper lip retractor is connected to the hinge spring by a maxillary occlusal arm. In yet another aspect, the upper lip retractor comprises an upper lip pull.

In another aspect, the lower lip retractor is in the shape of a lower dental arch. In another aspect, the lower dental arch has a width of about 45 mm to about 75 mm. In another aspect, the lower dental arch has a radius of about 20 mm to about 35 mm. In another aspect, the lower lip retractor comprises a well for receiving the lower lip. In another aspect, the lower lip retractor is connected to the hinge spring by a mandibular occlusal arm. In another aspect, the lower lip retractor comprises a lower lip pull.

In another aspect, the well comprises a first edge and a second edge. In another aspect, the first edge is about 5 mm to about 15 mm in height. In another aspect, the second edge is about 5 mm to about 15 mm in height. In yet another aspect, the first and second edge are connected by a well shelf having a width of about 5 mm to about 10 mm.

Another embodiment described herein is a method for taking orofacial photography in a patient in need thereof comprising: inserting the appliance described herein into the patient's mouth; retracting the upper lip to expose the upper gingiva; retracting the lower lip to expose the lower gingiva; photographing the patient's upper and lower teeth.

In one aspect, inserting the appliance comprises placing the upper lip into the upper lip retractor well. In another aspect, inserting the appliance comprises placing the lower lip into the lower lip retractor well. In another aspect, inserting the appliance comprises placing the maxillary occlusal arm into the maxillary upper vestibule space. In another aspect, inserting the appliance comprises placing the mandibular occlusal arm into the mandibular lower vestibule space.

In another aspect, the retracting the upper lip comprises pulling the upper lip pull upward. In another aspect, retracting the lower lip comprises pulling the lower lip pull downward.

In another aspect, separating the upper lip retractor and the lower lip retractor between about 35 mm to about 60 mm.

In another aspect, photographing of the patient's upper or lower teeth comprises photographing the respective dental arch.

Another embodiment described herein is an appliance comprising: a. an upper element; b. a lower element; and c. a hinge element; wherein the upper element and lower element each further comprise a handling element for allowing a patient to hold the appliance in the mouth; and wherein the appliance does not include elements for positioning the tongue.

In yet another embodiment described herein, is an appliance comprising: a. an upper lip retractor; b. a lower lip retractor; c. a hinge spring; wherein the hinge spring exerts an upward force on the upper lip retractor and a downward force on the lower lip retractor.

In one aspect, the upper lip retractor is in the shape of an upper dental arch. In another aspect, the upper dental arch has a width of about 40 mm to about 75 mm. In another aspect, upper dental arch has a radius of about 20 mm to about 35 mm. In another aspect, the upper lip retractor comprises a well for receiving the upper lip. In another aspect, the upper lip retractor is connected to the hinge spring by a maxillary occlusal arm. In another aspect, the upper lip retractor comprises an upper lip pull.

In another aspect described herein, the lower lip retractor is in the shape of a lower dental arch. In another aspect, the lower dental arch has a width of about 45 mm to about 75 mm. In another aspect, the lower dental arch has a radius of about 20 mm to about 35 mmm. In another aspect, the lower lip retractor comprises a well for receiving the lower lip. In another aspect, the lower lip retractor is connected to the hinge spring by a mandibular occlusal arm. In another aspect, the lower lip retractor comprises a lower lip pull. In another aspect, the well comprises a first edge and a second edge.

In yet another aspect described herein, the first edge is about 5 mm to about 15 mm in height. In another aspect, the second edge is about 5 mm to about 15 mm in height. In another aspect, the first and second edge are connected by a well shelf having a width of about 5 mm to about 10 mm.

In another embodiment described herein, is a method for taking orofacial photography in a patient in need thereof comprising inserting the appliance described herein into the patient's mouth; retracting the upper lip to expose the upper gingiva; retracting the lower lip to expose the lower gingiva; and photographing the patient's upper and lower teeth.

In one aspect, the inserting the appliance comprises placing the upper lip into the upper lip retractor well. In another aspect, the inserting the appliance comprises placing the lower lip into the lower lip retractor well. In another aspect, the inserting the appliance comprises placing the maxillary occlusal arm into the maxillary upper vestibule space. In another aspect, the inserting the appliance comprises placing the mandibular occlusal arm into the mandibular lower vestibule space. In another aspect, the retracting the upper lip comprises pulling the upper lip pull upward. In another aspect, the retracting the lower lip comprises pulling the lower lip pull downward. In another aspect, the method further comprises separating the upper lip retractor and the lower lip retractor between about 35 mm to about 60 mm. In another aspect, the photographing of the patient's upper or lower teeth comprises photographing the respective dental arch.

Another embodiment described herein is a method for taking orofacial photography in a patient in need thereof comprising: inserting a lip and cheek expansion device into the patient's mouth; retracting the upper lip to expose the upper gingiva; retracting the lower lip to expose the lower gingiva; wherein the lip and cheek expansion device comprises: i. an upper frame element forming a portion of a frame, the upper frame element including a left frame portion that posteriorly bends and a right frame portion that posteriorly bends ; ii. a lower frame element forming a portion of the frame, the lower frame element including a left frame portion that posteriorly bends and a right frame portion that posteriorly bends, the left side of the upper frame element posteriorly connected to the left side of the lower frame element, and the right side of the upper frame element posteriorly connected to the right side of the lower frame element; wherein the device is selectively positionable in a collapsed configuration and in an expanded configuration; wherein, in the expanded configuration, a central portion of the upper frame element is separated from a central portion of the lower frame element by an upper-to-lower dimension and the first left portion of the frame is separated from the right portion of the frame by a side-to-side dimension; wherein the collapsed configuration, the upper frame element deforms at one or more regions between the anterior portion and the posterior portion of the left side frame, the upper frame elements deforms at one or more regions between the anterior portion and the posterior portion of the right side frame, the lower frame element deforms at one or more regions between the anterior portion and the posterior portion of the first side of the frame, the lower frame element deforms at one or more regions between the anterior portion and the posterior portion of the right side of the frame, the upper-to-lower dimension is reduced, and the posterior frame element deforms to reduce the side-to-side dimension.

One or more aspects and embodiments may be incorporated in a different embodiment although not specifically described. That is, all aspects and embodiments may be combined in any way or combination.

### BRIEF DESCRIPTION OF THE DRAWINGS

FIG. 1: FIG. 1 is a schematic describing the appliance of the present disclosure, referred to as device 1.

FIG. 2: FIG. 2 is a top down view of the appliance of the present disclosure.

FIG. 3: FIG. 3 is a side view of the appliance of the present disclosure.

FIG. 4: FIG. 4 is a cross-sectional view of the appliance of the present disclosure.

FIG. 5 is another embodiment of the appliance of the present disclosure, referred to as device 2.

### DETAILED DESCRIPTION OF THE INVENTION

The present disclosure relates to a novel dental appliance and methods for orofacial photography. The dental appliance comprises means for retracting the upper and lower lips so as to fully expose the upper and lower front teeth and the upper and lower dental arch in order facilitate unimpeded photography of all aspects of tooth alignment during the treatment period.

In the following detailed description, various embodiments of the present disclosure will be described. For purposes of explanation, specific configurations and details are set forth in order to provide a thorough understanding of the embodiments. However, it will also be apparent to one skilled hi the art that the present disclosure may be practiced without the specific details. Furthermore, well-known features may be omitted or simplified hi order not to obscure the embodiment being described. It is further understood that features of the various embodiments may be combined with each other as would be understood by the skilled artisan. Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein may be used in practice or testing of the present disclosure. All publications, patent applications, patents and other references mentioned herein are incorporated by reference in their entirety. The materials, methods, and examples disclosed herein are illustrative only and not intended to be limiting.

As used herein, the articles “a,” “an,” and “the” are used herein to refer to one or to more than one (i.e., to at least one) of the grammatical object of the article. By way of example, “an element” may mean one element or more than one element.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In some embodiments, the terms “about” or “approximately” when preceding a numerical value indicates the value plus or minus a range of 15%, 10%, 5%, or 1%.

As used herein, the terms “individual”, “subject” and “patient” are often used interchangeably and refer to any human or animal that may be treated with the methods disclosed herein. Suitable subjects (e.g., patients) include humans, animals (such as mouse, rat, rabbit, or guinea pig), farm animals, and domestic animals or pets (such as a cat or dog). Non—human primates and human patients are included. In one embodiment, subjects may include human patients that are seeking dental or orthodontic treatment. As used herein, the term “patient” refers to a human subject that may receive oral, dental and/or orthodontic treatment for any disease or condition.

As used herein, “treatment”, “treat”, and “treating” refers to reversing, alleviating, mitigating, or slowing the progression of, or inhibiting the progress of, a disorder or disease, tooth decay, misaligned teeth, improper dental bite or oral conditions as described herein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6,4, 6.5, 6.6, 6.7, 6.8, 6.9, and 7.0 are explicitly contemplated.

During dental and/or orthodontic treatment, it may be necessary for a patient to take photos of their own teeth and send to the provider. In order to facilitate unimpeded imaging, the teeth should be fully and completely exposed and visible to a camera. The patient should be focused on holding the camera to obtain the orientation required for obtaining unimpeded images rather than manually retracting the lips to expose the teeth with one hand and holding the camera in the other. The presently disclosed dental appliance and methods provide means for retraction of the lips to fully and completely expose the teeth without requiring the patient to hold the lips or appliance in place.

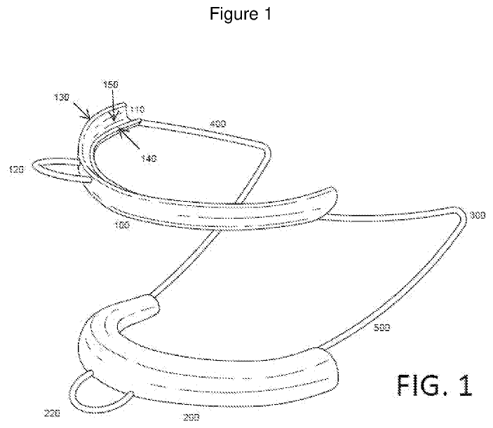

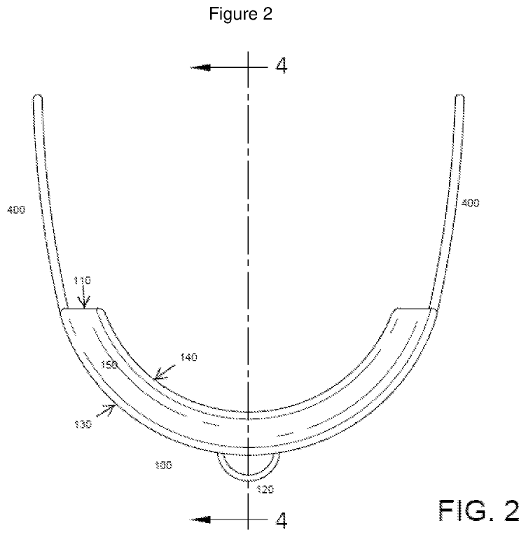

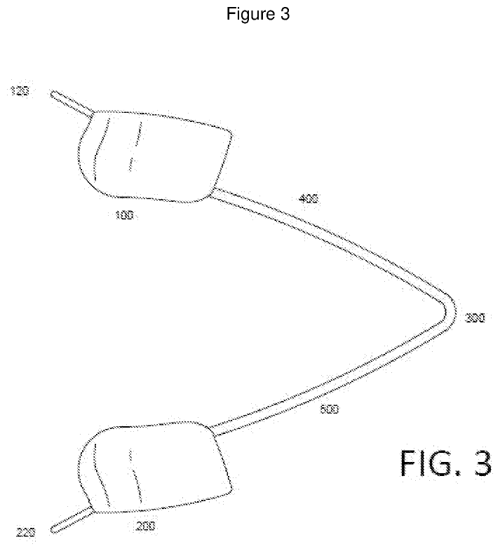

Referring now to the drawings, in which like reference numerals represent like parts throughout the several views, FIGS. 1-4 provide an appropriate starting point in a detailed discussion of the various embodiments of the present disclosure with respect to the dental appliance described herein. A dental appliance, device 1, is described in FIGS. 1-4. The appliance is a single unit but may be designed to be several different pieces assembled to create a single appliance. The appliance comprises an upper lip retractor 100 and a lower lip retractor 200 (also referred to herein as “upper and lower elements” respectively) connected by a hinge spring 300. The upper lip retractor 100 is connected to the hinge spring 300 by way of a maxillary occlusal arm 400 that extends from each end of the upper lip retractor. The lower lip retractor 200 is connected to the hinge spring 300 by a mandibular occlusal arm 500 that extends from each end of the lower lip retractor. The hinge spring may be either mechanically set to a specific position or may exert a tension and/or force to facilitate both the upward movement of the upper lip retractor (upward force) and the downward movement of the lower lip retractor (downward force).

Thus, one embodiment described herein is a dental appliance comprising an upper lip retractor; a lower lip retractor and a hinge spring wherein the hinge spring exerts an upward force up on the upper lip retractor and a downward force on the lower lip retractor to retract the upper and lower lips respectively.

In one aspect, the upper lip retractor is in the shape of an upper dental arch. The shape thus allows the appliance to follow the natural curve of the mouth comfortably once inserted into the mouth.

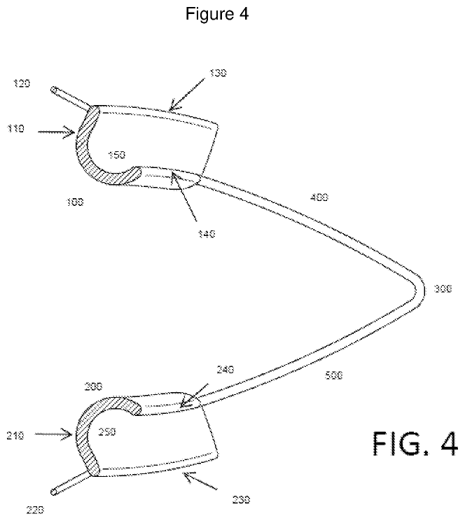

In another aspect the upper lip retractor is shaped to include a well 110. In one embodiment and as described in FIG. 4, the well may be slightly U-shaped such that when the appliance is inserted into the mouth the upper lip may rest inside the well. Thus, in one aspect the well is designed to receive and maintain the upper lip.

In yet another aspect, the well comprises a first edge 130. In one aspect the first edge comprises a height of about 1 mm to about 20 mm, about 5 mm to about 10 mm, about 7 mm to about 8 mm. In one aspect the first edge comprises a height of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. In one aspect, the first edge comprises a height of about 5 mm to about 15 mm.

In another aspect, the well comprises a second edge 140. In one aspect the second edge comprises a height of about 1 mm to about 20 mm, about 5 mm to about 10 mm, about 7 mm to about 8 mm. hi one aspect the second edge comprises a height of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. In one aspect, the second edge comprises a height of about 5 mm to about 15 mm.

In one aspect, the first and second edge comprise an equal height. In another aspect, the first edge comprises a height greater than the height of the second edge. In another aspect, the second edge comprises a height greater than the height of the first edge.

In another aspect the first and second edge are connected by a well shelf 150 where the lip sits once inserted into the well. In one aspect, the well shelf should be wide enough to comfortably fit an upper lip of any thickness. In another aspect, the well shelf has a width of about 1 mm to about 20 mm, about 5 mm to about 10 mm, about 7 mm to about 8 mm. In one aspect the well shelf comprises a width of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. In one aspect, the well shelf comprises a width of about 5 mm to about 10 mm.

In another aspect, the upper lip retractor comprises an upper lip pull 120. The pull is designed to be held by the patient for inserting the appliance in the mouth and may be used to exert an upward force to retract one or both lips from the surface of the teeth. Alternatively, the upward pull may be used to mechanically set the hinge spring to a desired position that retracts the upper lip.

In one aspect, the lower lip retractor is in the shape of a lower dental arch 200. The shape allows the appliance to follow the natural curve of the lower part of the mouth comfortably once inserted into the mouth.

In another aspect, the lower lip retractor is shaped to include a well 210. The well is slightly U-shaped such that when the appliance is inserted into the mouth the lower lip may rest inside the well. Thus, in one aspect the well of the lower lip retractor is designed to receive and maintain the lower lip.

In yet another aspect, the lower lip retractor well comprises a first edge 230. In one aspect the first edge comprises a height of about 1 mm to about 20 mm, about 5 mm to about 10 mm, about 7 mm to about 8 mm. In one aspect the first edge comprises a height of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. In one aspect, the first edge comprises a height of about 5 mm to about 15 mm.

In yet another aspect, the lower lip retractor well comprises a second edge 240. In one aspect the second edge comprises a height of about 1 mm to about 20 mm, about 5 mm to about 10 mm, about 7 mm to about 8 mm, In one aspect the second edge comprises a height of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. In one aspect the second edge comprises a height of about 5 mm to about 15 mm.

In one aspect, the first and second edge comprise an equal height. In another aspect, the first edge comprises a height greater than the height of the second edge. In another aspect, the second edge comprises a height greater than the height of the first edge.

In another aspect the first and second edge of the lower lip retractor well are connected by a well shelf 250 where the lip sits once inserted into the well. In one aspect, the well shelf should be wide enough to comfortably fit a lower lip of any thickness. In another aspect, the well shelf has a width of about 1 mm to about 20 mm, about 5 mm to about 10 mm, about 7 mm to about 8 mm. In one aspect the well shelf comprises a width of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm or about 20 mm. In one aspect, the well shelf comprises a width of about 5 mm to about 10 mm.

In another aspect described herein, the hinge spring exerts a tension force that separates the upper lip retractor and the lower lip retractor to an optimal distance for retracting the lips and photography of between about 20 mm to about 75 mm. In another embodiment described herein, the hinge spring may be mechanically adjusted further to maintain a desired distance between the upper lip retractor and the lower lip retractor by way of the patient using the upper lip pull and exerting an upward force or using the lower lip pull to exert a downward force (the upper and lower lip pulls may also be referred to herein as the “upper and lower handling elements” respectively).

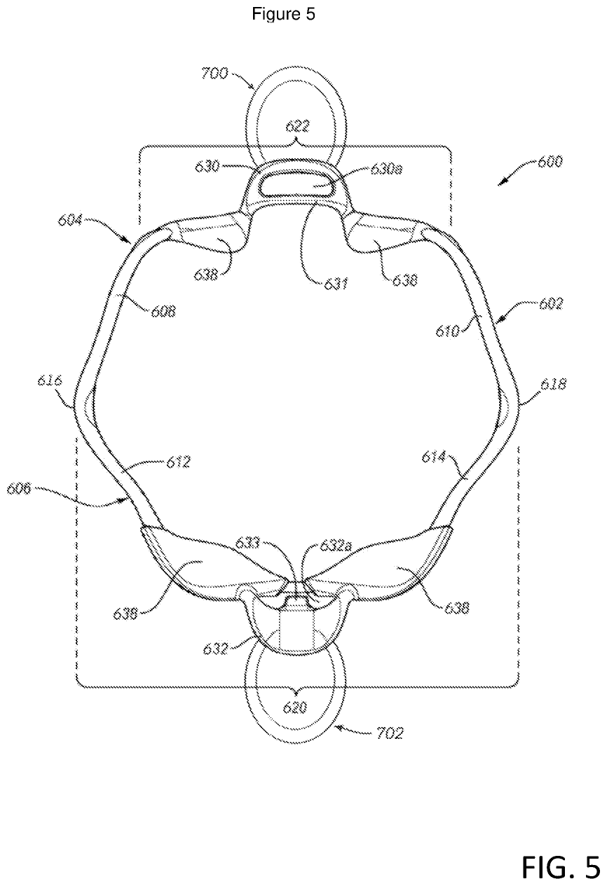

FIG. 5 depicts a second embodiment of the appliance of the present disclosure, which for ease of reference may be referred to as device 2. Device 2 includes a frame 602 that is selectively collapsible and expandable. Frame 602 may include an upper frame element 604, which may bear against and retract cheeks and/or lips from teeth of the upper dental arch, and a lower frame element 606, which retracts cheeks and/or lips from teeth of the lower dental arch, (the upper frame and lower frame elements may also be referred to herein as “upper and lower elements” respectively). Upper frame element 604 may include left frame portion 608 and right frame portion 610. Similarly, lower frame element 606 may include left frame portion 612 and right frame portion 614. Frame portions 608 and 612 may be joined to one another to form a v-shaped hinge 616, and right frame portions 610 and 614, may be joined to one another to form another v-shaped hinge 618 on an opposite side of frame 602.

A posterior width of upper 604 and lower 606 frame elements as defined between the right and left frame portions adjacent v-shaped hinges 616, 618 (e.g., the width from a vertex of angled hinge 616 to the vertex of angled hinge 618) may be greater than an anterior width of the upper 604 and lower 606 frame elements.

Both members 630, 632 may include a hollow portion 630a, 632a, respectively. Upper lip protecting member 630 may include a cross-member 631 adjacent portion 630a, which may serve to latch with a corresponding latch member 633 of lower lip protecting member 632. Lip protecting members 630 and 632 may curve over and extend the patient's lips during photography, extending outside the patient's mouth. Because of their extension outside of the mouth, they provide a convenient handle 700 or 702 (which may also be referred to herein as the “upper and lower handling elements” respectively).that can be gripped when removing or positioning the device.

When installed, device 2 pushes out on the lips and the cheeks simultaneously, retracting these soft tissues away from the dental arches.

The embodiments of the present disclosure advantageously may allow full closure of the patient's mouth, with the device installed. This is a distinct advantage over many existing cheek retractor devices, where full closure of the jaw is not possible. Because closure of the jaw is possible, the patient may insert the device, retract the cheeks and lips, bite down and take suitable photographs of the dental bite.

The appliance of the present disclosure may be made from any suitable material as commonly used for oral/dental/orthodontic appliances. Exemplary materials include both hard and soft polymers and plastics. Suitable polymeric materials may include but not be limited to styrenic block copolymer (SBC), a silicone rubber, an elastomeric alloy, a thermoplastic co-polyester elastomer, a thermoplastic polyimide elastomer, or a combination thereof (e.g., a blend of at least two of the listed polymeric materials).

The appliance may have a variety of physical properties suitable for the particular use. For example, physical properties such as hardness, ultimate tensile strength, elongation at break, tensile modulus, compression set and flexural modulus may each be specifically tailored for the particular application. The appliance may also be moldable such that the patient may be able to compress or expand the lip retractors to comfortably fit their lips. Alternatively, the appliance may have standardized physical properties designed for use with any patient.

Also described herein are kits that include the appliance of the instant disclosure. Appliances of the instant disclosure may be of a standard size such that they may be used by any patient or may be custom sized to fit a particular patient. Kits would include the appliance, directions on how to use, directions on how to clean and example images to be taken by the patient while using the appliance.

The appliance of the instant disclosure may be used for a variety of purposes, including visualizing a patient's teeth and gingiva for diagnosis and treatment, and allowing a patient to easily take photos of their own teeth for these purposes. Thus, one embodiment described herein is a method for taking orofacial photography in a patient in need thereof. The method comprising inserting the appliance of the instant disclosure into a patient's mouth; retracting the upper lip to expose the upper gingiva; and/or retracting the lower lip to expose the lower gingiva; and photographing the patient's upper and/or lower teeth, as applicable.

As a patient places the appliance in their mouth, the appliance should be positioned such that the upper and lower lip are fit into the well of each upper lip retractor and lower lip retractor respectively. Thus, in one aspect, inserting the appliance comprises placing the upper lip into the upper lip retractor well. Similarly, in another aspect, inserting the appliance comprises placing the lower lip into the lower lip retractor well.

Another aspect of the insertion step involves placing the appliance so that the maxillary occlusal arm fits into the buccal space. The buccal space may be understood to be the space inside the patient's cheek. In one aspect, inserting the appliance comprises placing the maxillary occlusal arm into the buccal space. In another aspect, inserting the appliance comprises placing the maxillary occlusal arm into the maxillary upper vestibule space. Alternatively, the insertion step may also place the appliance so the maxillary occlusal arm sits along the top surface of the upper dental arch. Thus, in another aspect, inserting the appliance comprises placing the maxillary occlusal arm along the top surface of the upper dental arch.

Another aspect of the insertion step involves placing the appliance so that the mandibular occlusal arm fits into the buccal space. The buccal space may be understood to be the space inside the patient's cheek. In one aspect, inserting the appliance comprises placing the mandibular occlusal arm into the buccal space. In another aspect, inserting the appliance comprises placing the mandibular occlusal arm into the mandibular lower vestibule space. Alternatively, the insertion step may also include placing the appliance so the mandibular occlusal arm sits along the top surface of the lower dental arch. Thus, in another aspect, inserting the appliance comprises placing the maxillary occlusal arm along the top surface of the lower dental arch.

Another aspect of the method of using the appliance of the instant disclosure is utilizing the lip pull on each lip retractor. The lip pull allows the patient to hold on to the pull while placing the appliance in the mouth. Although the hinge spring includes tension to keep the upper lip retractor and lower lip retractor separated, the patient may use the lip pull on each retractor to further retract the lips. Thus, in one aspect, retracting the upper lip comprises pulling the upper lip pull upward. In another aspect, retracting the lower lip comprises pulling the lower lip pull downward. In another aspect, the method comprises separating the upper and lower retractors a distance suitable for visualizing the patient's upper and lower dental arch, such as between about 35 mm to about 60 mm.

The retraction of each of the upper and lower lip comprises retracting the lips far enough so as to expose the patients upper and lower gingiva. Exposure of the gingiva ensures that the teeth are fully exposed so as to be able to observe all the teeth and visualize teeth alignment.

Once the appliance is inserted into the patient's mouth and the lips are retracted, the patient may proceed to orient any suitable camera to take images of the teeth. Suitable images include full and unobstructed views of the teeth including front and side views of the upper and lower teeth, as well as complete view of the upper and lower dental arch. Any combination of images may be taken to provide complete visualization of the patient's teeth to dentist. Images may be emailed to the dentist and used to determine diagnosis, prognosis, and further course of treatment plans.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. Those skilled in the art will readily recognize a variety of noncritical parameters that could be changed or modified to yield essentially similar results.

What is claimed is:

1. An appliance comprising:
a. an upper element;
b. a lower element;
c. a hinge element;
wherein the upper element and lower element each further comprise a handling element for allowing a patient to hold the appliance in the mouth; and
wherein the appliance does not include elements for positioning the tongue.

2. An appliance comprising:
a. an upper lip retractor;
b. a lower lip retractor;
c. a hinge spring; and
wherein the hinge spring exerts an upward force on the upper lip retractor and a downward force on the lower lip retractor.

3. The appliance of claim 2, wherein the upper lip retractor is in the shape of an upper dental arch.

4. The appliance of claim 3, wherein the upper dental arch has a width of about 40 mm to about 75 mm.

5. The appliance of claim 3, wherein the upper dental arch has a radius of about 20 mm to about 35 mm.

6. The appliance of claim 3, wherein the upper lip retractor comprises a well for receiving the upper lip.

7. The appliance of claim 2, wherein the upper lip retractor is connected to the hinge spring by a maxillary occlusal arm.

8. The appliance of claim 2, wherein the upper lip retractor comprises an upper lip pull.

9. The appliance of claim 2, wherein the lower lip retractor is in the shape of a lower dental arch.

10. The appliance of claim 9, wherein the lower dental arch has a width of about 45 mm to about 75 mm.

11. The appliance of claim 9, wherein the lower dental arch has a radius of about 20 mm to about 35 mmm.

12. The appliance of any claim 2, wherein the lower lip retractor comprises a well for receiving the lower lip.

13. The appliance of claim 2, wherein the lower lip retractor is connected to the hinge spring by a mandibular occlusal arm.

14. The appliance of claim 2, wherein the lower lip retractor comprises a lower lip pull.

15. The appliance of claim 6, wherein the well comprises a first edge and a second edge.

16. The appliance of claim 15, wherein the first edge is about 5 mm to about 15 mm in height.

17. The appliance of claim 13, wherein the second edge is about 5 mm to about 15 mm in height.

18. The appliance of claim 15, wherein the first and second edge are connected by a well shelf having a width of about 5 mm to about 10 mm.

19. A method for taking orofacial photography in a patient in need thereof comprising:
a. inserting the appliance of claim 2 into the patient's mouth;
b. retracting the upper lip to expose the upper gingiva;
c. retracting the lower lip to expose the lower gingiva; and
d. photographing the patient's upper and lower teeth.

20. The method of claim 19, wherein the inserting the appliance comprises placing the upper lip into the upper lip retractor well.

21. The method of claim 19, wherein the inserting the appliance comprises placing the lower lip into the lower lip retractor well.

22. The method of claim 19, wherein the inserting the appliance comprises placing the maxillary occlusal arm into the maxillary upper vestibule space.

23. The method of claim 19, wherein the inserting the appliance comprises placing the mandibular occlusal arm into the mandibular lower vestibule space.

24. The method of claim 19, wherein the retracting the upper lip comprises pulling the upper lip pull upward.

25. The method of claim 19, wherein the retracting the lower lip comprises pulling the lower lip pull downward.

26. The method of claim 19, further comprising separating the upper lip retractor and the lower lip retractor between about 35 mm to about 60 mm.

27. The method of claim 19, wherein the photographing of the patient's upper or lower teeth comprises photographing the respective dental arch.

28. A method for taking orofacial photography in a patient in need thereof comprising:
a. inserting a lip and cheek expansion device into the patient's mouth;
b. retracting the upper lip to expose the upper gingiva;
c. retracting the lower lip to expose the lower gingiva;
wherein the lip and cheek expansion device comprises:
i. an upper frame element forming a portion of a frame, the upper frame element including a left frame portion that posteriorly bends and a right frame portion that posteriorly bends;
ii. a lower frame element forming a portion of the frame, the lower frame element including a left frame portion that posteriorly bends and a right frame portion that posteriorly bends, the left side of the upper frame element posteriorly connected to the left side of the lower frame element, and the right side of the upper frame element posteriorly connected to the right side of the lower frame element;
wherein the device is selectively positionable in a collapsed configuration and in an expanded configuration;
wherein, in the expanded configuration, a central portion of the upper frame element is separated from a central portion of the lower frame element by an upper-to-lower dimension and the first left portion of the frame is separated from the right portion of the frame by a side-to-side dimension;
wherein the collapsed configuration, the upper frame element deforms at one or more regions between the anterior portion and the posterior portion of the left side frame, the upper frame elements deforms at one or more regions between the anterior portion and the posterior portion of the right side frame, the lower frame element deforms at one or more regions between the anterior portion and the posterior portion of the first side of the frame, the lower frame element deforms at one or more regions between the anterior portion and the posterior portion of the right side of the frame, the upper-to-lower dimension is reduced, and the posterior frame element deforms to reduce the side-to-side dimension.
